(19) United States
(12) Patent Application Publication
(10) Pub. No.: US 2016/0032239 A1
(43) Pub. Date: Feb. 4, 2016

(54) Deterministic Manufacturing Process For Creating 3D Living Tissues Based on 2D Directed Assembly And Origami Techniques

(71) Applicant: NORTHEASTERN UNIVERSITY, Boston, MA (US)

(72) Inventors: Carol LIVERMORE-CLIFFORD, Framingham, MA (US); Sangeeta BHATIA, Lexington, MA (US); Robert LANG, Alamo, CA (US); Roger ALPERIN, Walnut Creek, CA (US); Martin Luther CULPEPPER, Georgetown, MA (US); Majid Bigdeli KARIMI, Allston, MA (US)

(21) Appl. No.: 14/774,468

(22) PCT Filed: Mar. 12, 2014
(86) PCT No.: PCT/US2014/024905

§ 371 Date: Sep. 10, 2015

Related U.S. Application Data
(60) Provisional application No. 61/777,651, filed on Mar. 12, 2013.

(51) Int. Cl. C12N 5/00; A61L 27/18; A61L 27/38; C12N 5/071; C12N 5/077

(52) U.S. Cl. CPC C12N 5/0068; C12N 5/069; C12N 5/0656; C12N 5/067; A61L 27/3895; A61L 27/3891; A61L 27/18; C12N 2533/30; A61L 2400/18

(57) ABSTRACT

A method of forming 3D engineered tissues by providing a 2D scaffold material comprising a plurality of fold locations and a plurality of cell assembly sites, assembling cells into the cell assembly sites and folding the 2D scaffold material along the fold locations to form a 3D scaffold structure. Tissues formed by the method.

### CROSS-REFERENCE(S) TO RELATED APPLICATIONS

The present application claims the benefit of U.S. Provisional Patent Application No. 61/777,651, filed on Mar. 12, 2013, the content of which is hereby incorporated by reference herein in its entirety.

### TECHNICAL FIELD

This technology relates generally to methods for producing 3D living tissues based on 2D directed assembly and origami techniques.

### BACKGROUND

Improved engineering of human tissues has been identified as part of the Grand Challenge of personalized medicine by the National Academy of Engineering (NAE). Its benefits are simple: human tissue engineering can save lives. Tissue engineering can supplement the supply of organ transplants—which have been limited by the supply of donor organs, leading to thousands of deaths every year—as well as acting as a tool for screening new medical therapies before any human testing takes place.

Human tissue engineering can take place either within the body (in vivo, also termed regenerative medicine and often performed using stem cells) or outside the body (ex vivo). Ex vivo tissue engineering is very desirable because it offers the potential to develop the supply of organs and other tissue needed to meet the current demand, but it is also incredibly complex. Currently the state-of-the-art cannot produce human tissue fast enough (i.e. insufficient throughput), cannot in general produce complex human tissue suitable for transplantation or medical therapy testing (i.e. insufficient control), and it cannot produce it in a way that can be done to meet demand (i.e. insufficient scalability). It would be desirable to create a new way of structuring complex ex vivo engineered human tissue with excellent throughput, control, and scalability.

### SUMMARY

These and other aspects and embodiments of the disclosure are illustrated and described below.

This disclosure describes methods of forming 3D engineered tissues using a 2D scaffold material. In accordance with one aspect, the 2D scaffold material includes a number of fold locations and a number of cell assembly sites. Cells are assembled into the cell assembly sites and the 2D scaffold material is folded along the fold locations to form a 3D scaffold structure. In particular embodiments, the cells can be assembled using templated assembly by selective removal (TASR).

In accordance with certain aspects, at least some of the cell assembly sites are in a pattern that results in predefined structures in the 3D scaffold when the 2D scaffold material is folded into the 3D scaffold structure. Examples of predefined structures include, but are not limited to, vascular pathways. The vascular pathways can be positioned in the plane of the folded scaffold or located to cross between folded sheets.

In accordance with certain embodiments, more than one type of cells can be used to provide certain functionality to tissues produced in accordance with the method disclosed herein. In a particular embodiment, the cells include endothelial cells, hepatic cells, and fibroblasts.

The scaffold material typically is a biocompatible and biodegradable polymer. In some embodiments, the 2D scaffold material comprises thinned regions of the scaffold to facilitate bending. The 2D scaffold material may also include integrated actuators to induce partial or complete folding of the 2D scaffold without touching it. The 2D scaffold material may also include a surface coating to promote cell attachment, such as collagen. In accordance with some aspects, the scaffold structure may include a fold pattern resulting in a low number of degrees of freedom, or a single degree of freedom, so that the 2D scaffold folds with minimal contact with external objects.

The present application also relates to a tissue unit produced in accordance with the described method and larger scale tissues combining a number of tissue units in a modular fashion.

These and other aspects and embodiments of the disclosure are illustrated and described below.

### BRIEF DESCRIPTION OF THE DRAWINGS

The invention is described with reference to the following figures, which are presented for the purpose of illustration only and are not intended to be limiting.

In the Drawings:

FIG. 1A illustrates the concept of through holes for creating vascular pathways through folded layers;

FIG. 1B is a zoomed-out view of a creased scaffold sheet;

FIG. 1C shows a half-folded scaffold;

FIG. 1D shows one embodiment of a folded sheet structure with through-holes aligned to form blood flow paths;

FIG. 1E shows another embodiment of a folded sheet structure with through-holes aligned to form blood flow paths;

FIG. 2A illustrates some of the steps for assembling cells on a 2D scaffold material in accordance with one embodiment;

FIG. 2B provides an optical microscope image of 22 μm and 12 μm cells assembled into an array;

FIG. 3 illustrates a paper version of the Miura-ori;

FIG. 4A provides a crease pattern consisting of 3 units;

FIG. 4B is a photograph of a top view of the folded structure from FIG. 4A;

FIG. 4C is a photograph of a bottom view of the folded structure from FIG. 4A;

FIG. 5A is a schematic diagram of a folded sheet with folds forming quasi-planar hepatic cords;

FIG. 5B illustrates a zoomed-in conceptual cross-sectional diagram of a folded scaffold sheet patterned with perforations and through-holes and coated with hepatic cells (large cells) and endothelial cells (small cells) to form blood flow paths (arrows);

FIG. 5C is a diagram of alternate, accordion-folded architecture in which blood flows through inter-layer through holes;

FIG. 5D provides a diagram of direct “cinnamon roll” architecture in which blood also flows through inter-layer through holes;

FIG. 5E shows a section of a fold pattern that produces a hexagonal array of the quasi-cinnamon roll structure shown in FIG. 5F;

FIG. 5G is a fold pattern for a more complex origami structure yielding hexagonal symmetry; and

FIG. 5H shows the hexagonally symmetric fold of FIG. 5G implemented in paper.

### DETAILED DESCRIPTION

It will be appreciated that while a particular sequence of steps has been shown and described for purposes of explanation, the sequence may be varied in certain respects, or the steps may be combined, while still obtaining the desired configuration. Additionally, modifications to the disclosed embodiment and the invention as claimed are possible and within the scope of this disclosed invention.

Tissues cannot survive without both an adequate supply of nutrients and oxygen, and removal of metabolic byproducts. Vascular networks are critical to providing these functions in most human tissues, but effective vasculature has proven difficult to replicate in engineered tissues. A seemingly simple solution would be to seed cells onto a scaffold and let the developing tissue form its own vasculature, but the resulting networks are not sufficiently well-organized to supply the tissue. In fact, studies have shown that tissue function can even depend on its microstructure. For example, including multiple cell types in engineered tissues has been shown to improve the function of implanted tissue, and the microvascular structure of engineered heart tissue has been shown to affect its function after implant.

The present application discloses a tissue engineering methodology (the OATH process) based on directed assembly and origami folding techniques. The described system enables the rapid, parallel generation of fully 3D tissues with appropriate microstructure and effective vasculature by bringing together four critical elements from tissue engineering and other fields: (i) directed assembly to selectively locate multiple cell types in 2D; (ii) origami-based folding to translate 2D cell assemblies into 3D building blocks (and ultimately complete structures); and (iii) conventional methods of tissue engineering to ensure biologically-valid designs and processes, as well as assessment and validation of the results. Origami offers an ideal solution to the challenge of engineering tissues that are both microstructured and large in overall size because it enables a level of substructure that can neither be microscopically patterned on individual sheets, nor macroscopically assembled from component parts.

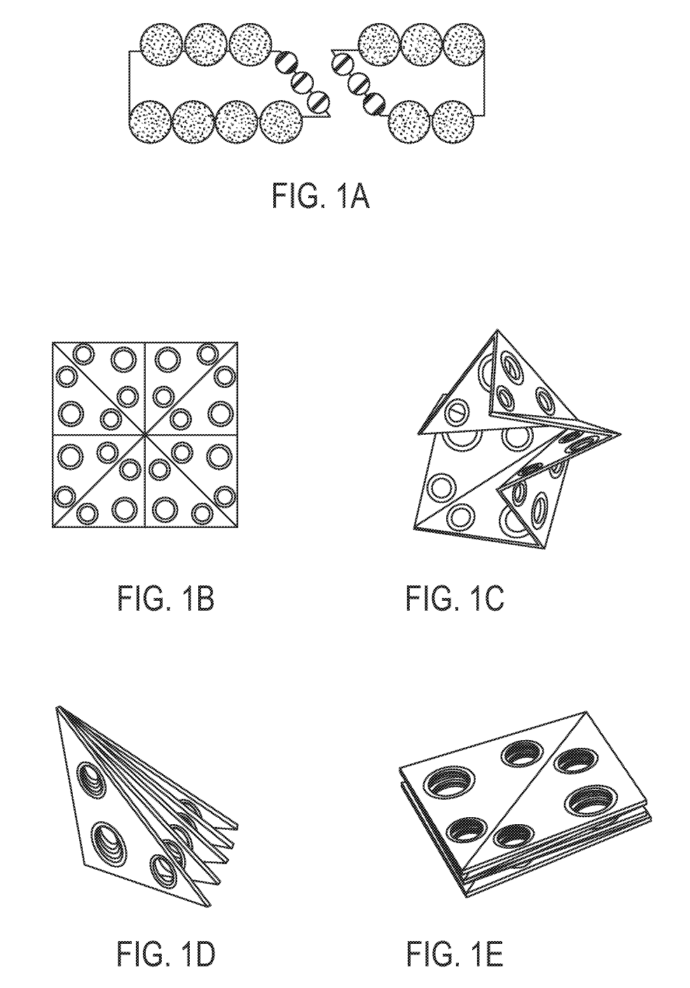

FIG. 1 illustrates one embodiment of the disclosed origami tissue concept. FIG. 1A illustrates the concept of through holes for creating vascular pathways through folded layers. FIG. 1B is a zoomed-out view of a creased scaffold sheet and FIG. 1C shows a half-folded scaffold. Two embodiments of a folded sheet structure with through-holes aligned to form blood flow paths are provided in FIGS. 1D and 1E.

In accordance with certain aspects, the disclosed method for engineering tissue is a two-step process with multiple sub-steps. Although the method is applicable to a wide variety of vascular tissues with or without symmetry, the example of hepatic (liver) tissue will be used to illustrate the process. First, cells and/or microtissues (small spheres containing cells and biomaterial) are assembled into pre-defined locations on a 2D biodegradable scaffold sheet (FIG. 1A). For the case of hepatic tissue, populations of hepatic cells (potentially also with fibroblasts) are assembled into regions that will ultimately be occupied by hepatic cells, and the cells that form vasculature (e.g., endothelial cells) are assembled into regions that will ultimately be occupied by vasculature. Wherever the vasculature is in the plane of the folded sheet (i.e. blood flows parallel to the sheet), endothelial cells are patterned on the sheet's surfaces. Wherever the vasculature crosses between folded sheets (as is illustrated conceptually in FIG. 1), endothelial cells are arranged around the periphery of what will ultimately become blood vessels. Second, the sheet is folded to form a 3D scaffold (FIGS. 1B-D). Since the cells have already been placed on the 2D scaffold sheet, they are pre-seeded in their appropriate locations in the 3D folded structure. Over time the cells coalesce, biomaterial degrades and remodels, and the scaffold biodegrades, leaving just the tissue.

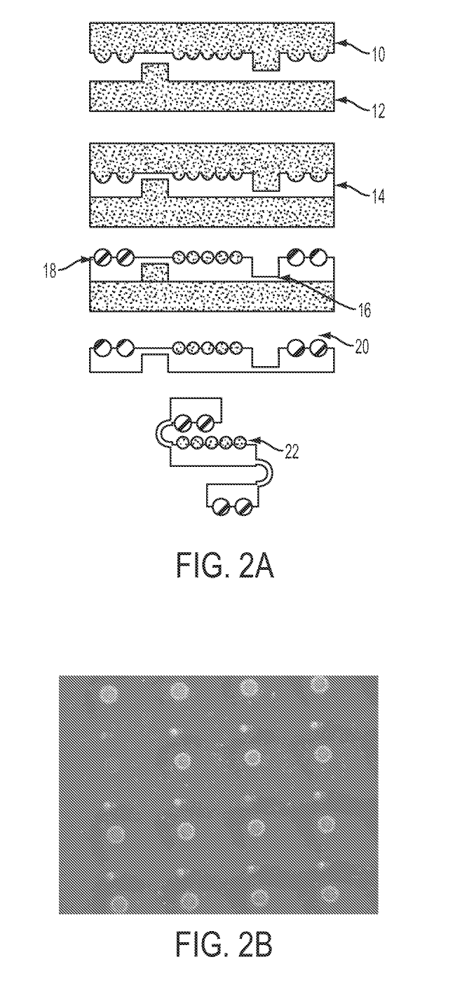

One embodiment of an assembly process is illustrated in FIG. 2A. Master molds 10, 12 from which polymer sheets 14 with the desired topography can be formed are created and sterilized. The thin polymer sheet 14 is molded to define hinges 16 and cell assembly sites 18. In the embodiment shown, the polymer sheet is cleaned while attached to the rigid substrate 12. Cells 20, 22 are assembled into sites 18 onto the polymer sheet and organized by type. The polymer sheet 14 is released from the substrate 12 and folded either manually or by triggering actuated folding. The assembly process may also include other steps not shown in this embodiment, such as adding surface layers, for example to promote adhesion of cells, sterilization, and the use of release layers to get the polymers off of the mold.

A key concept of the proposed method is its optional use of “modular” origami to solve the major problems of tissue engineering (throughput, control, and scalability). Most useful tissues require detailed structure at the microscopic level while taking form on a macroscopic scale that is orders of magnitude larger. It is extremely difficult to make the fully 3D microstructure of a large tissue directly. For example, assembly of macroscale components cannot produce the necessary microstructures; and 3D printing offers fine spatial control, but printing large tissues with a serial process is slow, requires specialized equipment and materials, and exposes cells to detrimental shear forces. On the other hand, folding a single 2D surface into a fully 3D tissue structure would require a gargantuan starting sheet. (To form even 1 in³ or 15 cm³ of tissue from a scaffold 100 μm thick would require a starting sheet size of about 40 cm×40 cm.) In accordance with certain aspects as disclosed herein, modular origami solves this problem. Folding is used to create modular building blocks from individual sheets that fold or self-fold to give the desired microscopic structure. The modularity provides improved control of larger, deterministically-designed blocks.

In accordance with one aspect, cells are assembled on the 2D scaffold using a directed assembly process that provides assembly selectivity via a combination of surface topography and ultrasound at frequencies similar to those used in medical applications. This assembly technique allows one to selectively place both individual cells (such as hepatocytes, fibroblasts, and endothelial cells for vasculature) and hepatic microtissues onto sterilized, biodegradable 2D scaffold sheets. The sheets may be pre-molded not only to promote selective cell and microtissue assembly, but also to form the structures necessary to ensure accurate folding (e.g., flexures representing origami crease patterns and through holes for vascular pathways). The scaffold sheets may also he coated with materials to promote cell adhesion, such as collagen or poly(ethylene glycol) (PEG). Other forms of directed cell assembly, such as sequential assembly of different cell types, can also be used to assemble cells on the 2D scaffold material.

Once the cells are assembled on the scaffold sheet, the sheet is folded to form a compact 3D structure in which the features of the individual layers align to create the intended structures in the 3D tissue. For example, a vascular pathway in the plane of the folded sheets, such as would mimic quasi-planar hepatic sinusoids, can be defined as a space between endothelial cells patterned on adjacent sheets. Alternatively, a blood vessel that crosses a series of folded layers can be comprised of through-holes in adjacent folded layers, lined with endothelial cells and aligned to create a blood vessel in the desired direction. Blood flow will he preferentially directed through flow channels that are designed for lower flow resistance. Latching of adjacent layers can act to enforce the as-designed geometry.

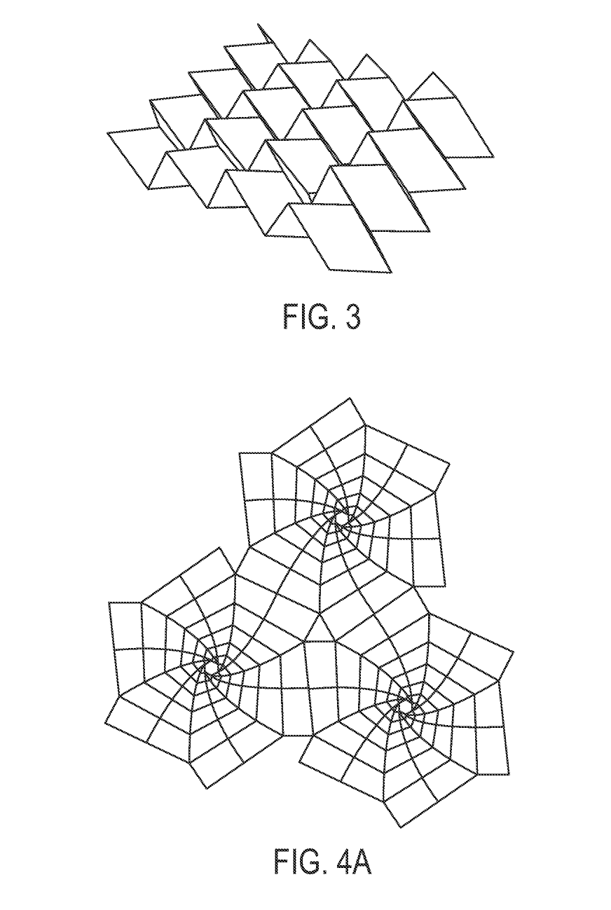

In accordance with certain embodiments, creases are pre-formed in the 2D scaffold sheet that correspond to a “minimal actuation” (requiring little intervention) fold pattern. A particularly useful pattern would be one that has 1 degree-of-freedom (1-DOF). Many 1-DOF fold patterns exist, and one well known example is the Miura-ori fold. A fold pattern with 1-DOF or a few degrees-of-freedom may he actuated quickly and simply by probing with a small number of sterile contacts. The Miura-ori is the best-known structure that exhibits the desired property of cross-structural actuation from a small number of actuation points, but there are many structures that can be drawn upon from the world of origami. The Miura-ori structure is shown in FIG. 3.

In accordance with another embodiment, self-folding actuators can be integrated with the 2D scaffold so that little or no external contact is required. Self-actuated scaffold sheets should be maintained in a fiat configuration during cell assembly and be actuated or released to fold only after cells are in place. The pattern of a self-actuated scaffold must be designed to fold correctly under the implemented release conditions: i.e., all at once for an instantaneous folding actuation, or sequentially from the edge to the center if the scaffold releases from the outside in, as when a sacrificial layer dissolves. A key advantage to the origami approach is that folding results in a marked increase in bending stiffness as compared with the stiffness of a single sheet. Increased stiffness limits deformation under loading (gravity or applied force), which in turn helps retain the alignment of structures on neighboring layers.

In accordance with certain embodiments, the present application provides methods for forming biomimetically-microstructured, vascularized tissues using high throughput techniques. Origami's inherent ability to pattern fine substructure offers a unique opportunity to achieve both biomimetic microstructure and large overall tissue sizes that are relevant for medical and biomedical applications. It is important to note that structuring the tissue is just part of the process. After cell assembly and scaffold folding, the structure must coalesce and remodel into fully-functional tissue under appropriate environmental stimuli, and its structure, viability, and metabolic functioning must be documented.

A particularly useful method for assembling cells on the 2D scaffold is template assembly by selective removal (TASR), which achieves selective placement of biological cells and/or inorganic objects through a combination of quantitative 3D shape matching and fluidic forces. When the topography of the surface matches the 3D shapes/sizes of the components/cells to be assembled, the increased surface interaction promotes component or cell assembly in that location. Fluidic forces provided by a very high frequency (MHz-range), ultrasonic excitation act to remove all but the components and cells that are best matched to the surface topography, ensuring selective assembly. The assembly is rapid and scalable, typically completing in 3-5 minutes independent of the number of components to be assembled.

Although the discussion herein relates to the use of the cells that comprise the liver, it should be understood that the method described herein is not limited to any particular cells or group of cells or to the creation of any particular type of tissue or of tissue with any particular function. Liver (hepatic) tissue is organized into subunits called “lobules”, each about 1-2 mm in size. Each lobule has an irregular hexagonal cross-section with approximately six-fold symmetry. Perpendicular to the cross-section, each lobule is extruded to form a roughly hexagonal prism with non-planar caps. Blood enters each lobule through six inlets organized around the lobule's periphery and fed by the portal vein and hepatic artery. Blood flows radially to the center of the lobule and exits through the central vein. Lobules are arranged in a rough array and connected to common vascular inlets and outlets.

The primary functional cells (parenchymal cells) of the liver are hepatocytes, which range from about 12 μm to 25 μm in diameter. Within each lobule, the hepatocytes are organized into hepatic cords, thin sheets of one or a few cells thick that branch and interconnect with each other. The hepatic cords are separated by sinusoids (hepatic vasculature), about 35 μm wide spaces through which blood flows. A perforated layer of endothelial cells (which at about 8 μm to 12 μm in diameter are smaller than hepatocytes) called the fenestrated endothelium shields the hepatic cords from the main blood flow and its accompanying shear stresses while permitting plasma to permeate the layer and wash directly over the hepatic cells for transfer of nutrients and other species. Connective tissue including fibro-blasts (10-15 μm cells that create extra cellular matrix (ECM) and collagen) lies between the endothelium and the hepatocytes.

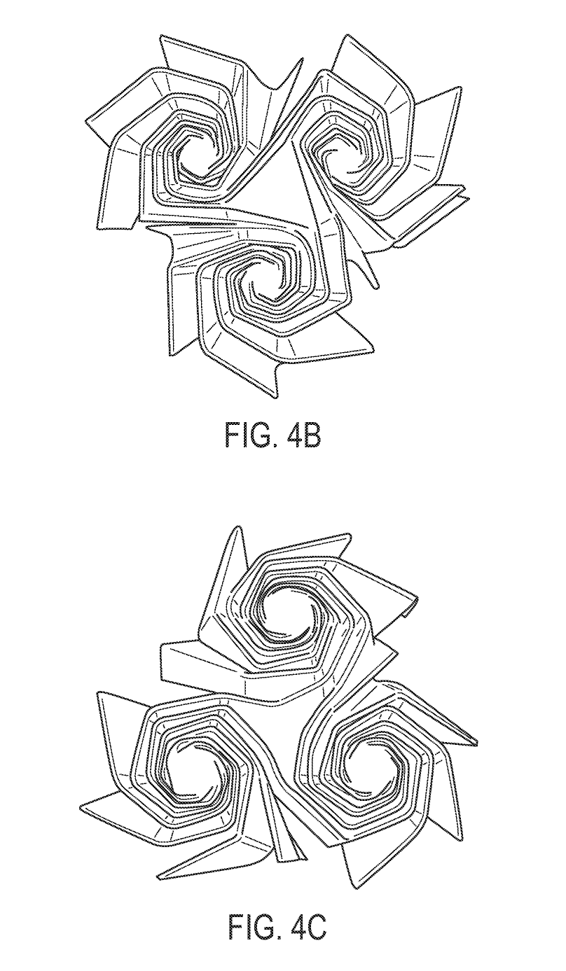

The hepatic tissue units can be designed to reflect the approximately six-fold symmetry of native liver lobules. This can be accomplished by creating hexagonal subunits, analogous to the hexagonal liver lobules, or by creating alternate subunits that reflect the lobules' symmetry. FIG. 4A shows a crease pattern consisting of 3 units, plus two photos (FIGS. 4B and 4C) of the folded shape (top and bottom). The unit tiles, so one can make arrays of arbitrary size, and there are tunable parameters for widths of the channels, height-to-spacing aspect ratio, etc. Each corner of the crease pattern includes a pair or up and down folds to form the folded shape.

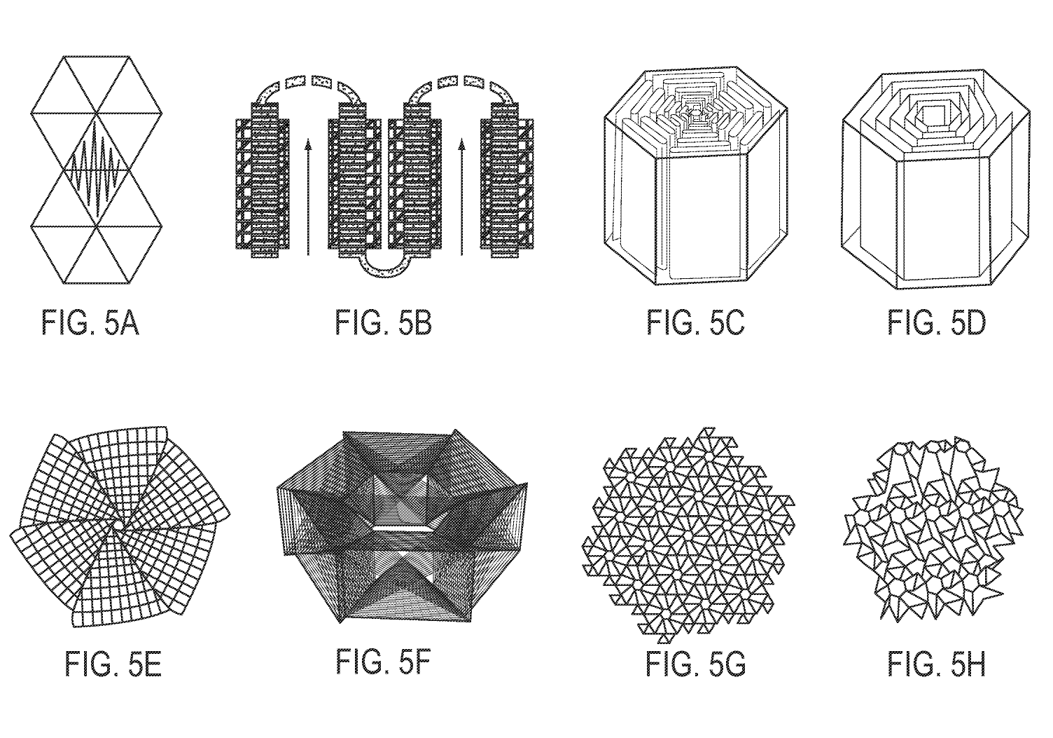

The sinusoids and the hepatic cords that separate them are not perfectly planar, but the local planarity that they exhibit between intersections is similar to the locally flat regions of a folded origami sheet. One approach to generating origami-folded, liver lobules is to leverage this structural similarity by orienting the folds radially, so that blood flows parallel to the folds (FIGS. 5A and 5B). Alternatively, the origami folds may be oriented approximately circumferentially, so that radial blood flow travels primarily through perforations in the origami sheets (FIGS. 5C through 5F). FIG. 5A shows a conceptual illustration of implementing radial blood flow in the partial-planar spaces between folded layers, for example as defined by a variation on the Miura-ori fold or as shown in FIG. 4. The layered structure of folds like the Miura-ori offers inherent matching to the quasi-planar sinusoid and hepatic cord structures of FIG. 4 and is shown conceptually as filling the “functional unit” parallelogram indicated in FIG. 4B. Multiple functional units are then tiled together in a modular fashion to form larger generated hepatic tissues.

FIG. 5B shows a simplified conceptual illustration of how cells can be patterned on the folded sheets of FIG. 5A to replicate the microstructure of liver lobules, with hepatic cords shielded from blood flow in the sinusoids by perforated layers of endothelial cells. In one aspect, the polymer scaffold is patterned with hepatic and endothelial cell assembly sites on opposite sides of the sheet, scaffold perforations to allow flow to the hepatocytes during scaffold resorption, and a set of thinned flexural joints to act as creases. Blood flows between layers of endothelial cells as indicated by the arrows in the figure, washes the hepatic cells through the perforations, and exits to the central vein through wide through-holes in the scaffold sheet. In accordance with one aspect, the structure of hepatocytes, endothelial cells, and blood flow pathways can be created by assembling cells on both sides of the scaffold sheet as shown in FIG. 5B, but a structurally similar structure with less spatial resolution may be defined by assembling cells on a single side of the scaffold sheet.

FIGS. 5C-5F illustrate alternate concepts for how the symmetries of the fold pattern and the tissue can be leveraged to create a tissue assembly concept. In these concepts, the scaffold runs perpendicular to the radial blood flow path, so that through holes in the scaffold sheet enable blood flow. In FIG. 5C, the scaffold is accordion-folded to create half of or a whole functional unit; in FIG. 5D, the scaffold is “rolled up” like a cinnamon roll at the joints to create the entire hexagonal lobule at once. In FIGS. 5E and 5F, the crease pattern folds a structure with symmetry like the cinnamon roll, with the additional advantage that the crease pattern may be extended to create multiple hexagonal units from a single larger sheet.

### Scaffold Material Selection

The function of the scaffold is to provide structure to the system during its formation and to degrade thereafter. The scaffold material should therefore be biocompatible and biodegradable on a practical timescale, with the scaffold material and its degradation products being harmless to the tissues. The materials should be patternable by molding, printing, and/or lithography to enable the creation of cell and microtissue assembly sites, flexural joints, and alignment features. Scaffolds should be sterilizable after manufacture, tolerating for example a temperature of 124° C. at 2 atm pressure for 15 minutes without significant geometry modification. The scaffold material should also be stiff enough to avoid unintended deformation but flexible enough to create bending flexures. (The quantitative constraints on stiffness reflect a combination of material stiffness and dimensions. For comparison, the stiffness of paper is on the order of a few GPa, and typical paper thickness is on the order of 50-100 urn.) The failure strain of the scaffold material must be large enough to prevent flexure breakage during folding. (A maximum strain of 50% permits bending to a radius of curvature equal to the flexure's thickness; lower strains require larger radii.) Finally, the scaffold material should be compatible with the manufacture of self-folding actuators that can pre-bias the fold directions (e.g., stressed bilayer actuators).

A diversity of biocompatible, biodegradable polymers has been created for medical applications and tissue engineering that may be useful in accordance with the present application. Examples of suitable biodegradable, biocompatible polymers are disclosed in U.S. Pat. No. 6,784,273, the content of which is hereby incorporated by reference. A particularly useful material is BDI-BDO-BDI-BDO-BDI/PCL, a thermoplastic polyurethane with Young's modulus of up to 0.1 GPa, >500% failure strain, and an ability to be molded by heating. (BDI is 1,4-butanediisocyanate, BDO is 1.4-Butanediol, and PCL is polycaprolactone, all of which degrade into benign species that occur naturally in the body.) The BDI segments speed up degradation, with full resorption observed in less than 3 months. Also useful are members of the poly(polyol sebacate) (PPS) family of chemically cross-linked polymers, which are well-studied, rapidly resorbable and can be patterned in the prepolymer stage. Examples include poly(sorbitol sebacate) (PSS), with a Young's modulus of up to 0.3 GPa, failure strains between 10 and 50%, and poly(maltitol sebacate) (PMtS).

In accordance with certain embodiments, the basic scaffold materials can be layered with bio-compatible material to create bilayer stress gradients and bias the folding direction and/or self-fold the scaffolds. A particularly useful material is polysuccinimide, which is stable in water but hydrolyzes in many liquids (including water and biological buffer solution) to form polyaspartic acid, which then swells to create a residual stress.

In origami tissue, the fold lines or creases may be compliant flexures In accordance with one aspect, the flexures may comprise living hinges, thinned regions of the scaffold that localize bending to the creases.

The methods described herein may also make use of interlocking alignment/latching elements to enforce relative alignment of the folded elements. These alignment elements facilitate correct alignment and maintaining final positions in a large network of compliant elements.

Based on the 3D tissue design and origami architecture, a 2D scaffold can be designed. The design may include crease locations, any alignment features, and the cell/microtissue assembly sites. The origami layout can be accomplished using a combination of conceptual origami design and computational methods. For the simplest cases, the locations of the cell types (and other features, such as through holes) may be set by the designer's knowledge of the structure's symmetry. In more complex 3D to 2D cases, the locations of features can be computationally mapped from the 3D structure to its 2D precursor sheet.

The 2D scaffold sheets will be designed to form the necessary topography for the assembly and origami processes; the features must be compatible with the intended scaffold manufacturing process and precision design considerations. Because the cell assembly process relies on shape matching, the sheet's topography must match the shapes and sizes of the cells/microtissues to be assembled at each location. In accordance with one aspect, a pattern can be created with hemispherical holes matched to the cells'/microtissues diameters in the locations where the individual elements are to assemble. The diameters of hepatocytes and endothelial cells are sufficiently different that excellent assembly selectivity should be obtained with appropriate choice of features sizes. For a greater degree of selectivity, or to pattern populations of both hepatocytes mixed with fibroblasts, microtissues of up to 100 μm in diameter may instead be assembled to template the hepatic tissue.

For some origami tissues, the cell pattern may be defined on only one side of the scaffold; for other structures, cell sites may be defined on both sides and potentially lining the through-holes. The living hinges can be implemented as thinned regions in the final scaffold sheet, and through-holes will mark locations where blood vessels cross the scaffold sheet, both for microvasculature and to provide the larger-scale vascular connections through which flow is supplied to the tissues.

An original master pattern can be microfabricated in silicon, SiO₂, glass, or polymers (e.g. SU-8 epoxy, polyurethane, or poly dimethyl siloxane (PDMS)) to create the assembly sites and folding crease features. If the master pattern is not an inverse (i.e., if it has holes where the final scaffold will have holes), then it must be replicated twice in order to create the final scaffold. The first replica will be an inverse replica (with bumps where the master had holes), and the second replica will be again have holes where the master pattern had holes. Robust inverse replicas of the masters (with bumps instead of holes at the assembly sites) can be molded of polymers, such as relatively rigid polyurethane. Finally, scaffolds with holes at the assembly site locations can be molded from the inverse replicas. If the master pattern is an inverse, then it must only be replicated once to create the final scaffold. In its simplest form, molding enables features to be patterned on just one surface of the scaffold, for example by spin casting the scaffold material. To pattern both sides of the scaffold, the material can be molded between upper and lower aligned, patterned plates (similar to nanoimprint lithography with a release layer). Procedures for molding the proposed scaffold materials are known to one of ordinary skill in the art. The creation of actuated scaffolds may additionally require photolithography to pattern the actuators, if present.

In accordance with particular embodiments, it is an important aspect of the scaffold manufacture/cell assembly process that the scaffolds be sterile when the cells are assembled and remain sterile thereafter. Examples of processes that may be used include: (i) release the scaffolds from the master mold prior to sterilization, coating with any necessary surface layers, and cell assembly and (ii) sterilize both scaffold and master mold, coat with any necessary surface layers, assemble the cells, and passively release the scaffold from the master pattern, e.g., by dissolving a sacrificial layer such as sucrose or poly vinyl alcohol (PVA). Some cells, such as hepatic cells may need a surface layer to stick, while the cells, such as fibroblasts, may not.

In accordance with one aspect, folding after cell assembly may be accomplished by the direct interaction of sterile probes with a low-DOF structure; this approach enables folding while minimizing contact with the tissue scaffold. In accordance with other aspects, folding may be accomplished in whole or in part by self-folding actuators. For example, in the case where polysuccinimide is hydrolyzed to trigger self-folding, the difference in time scales of assembly and hydrolysis can ensure that the cells are assembled before folding occurs Alternatively, a delayed structural release mechanism (such as the progressive dissolution of a sucrose release layer) can delay the folding until after cells assembly is complete.

The cells and microtissues can be assembled onto 2D sheets using directed assembly. The microtissues may be self-contained spheres containing cells (e.g., hepatocytes and fibroblasts that support hepatic function) and biomaterials that are created by photopatterning carrier material such as polyethylene glycol-diacrylate (PEG-DA) The directed assembly technique selectively places cells and/or microtissues through a combination of quantitative shape matching and very high frequency (MHz-range) ultrasonic excitation. The assembly is rapid, typically completing in 3-5 minutes.

Double-sided assembly may be realized through a two step process of assembling one side and then the other because gravitational forces are of minimal importance to near neutrally buoyant cells that are being stirred by a high frequency ultrasonic excitation.

The folding of cell-seeded tissues can be carried out in a benign environment (e.g., cell culture medium) After folding, flow can be driven from the patterned, larger-scale vascular connections, through the finer-scale folded vascular network of the tissue to supply nutrients and remove waste products. Flow can be maintained continually while the cells coalesce, biomaterial in the microtissues degrades and remodels, and the scaffold biodegrades, leaving just the tissue.

The tissue can be characterized during assembly and at intervals thereafter using conventional techniques for assessing cell populations and engineered tissues.

The creation of large mass, microstructured tissues can be obtained by modular stacking. Modular stacking also offers several different avenues for implementation. The most ambitious approach would be to directly stack tissue units together, aligning their vasculature directly in the process. At the opposite extreme, the simplest approach is to stack tissue units in alternating layers with separate vascular plena, so that the vasculature of each unit is fed by a common source. Origami offers the most intriguing approach, and arguably the approach that offers the largest degree of control over the final micro and macro scale structures. In this approach, individual folded tissue units are assembled onto a planar layer into which all of the necessary, larger-scale vascular connections have been patterned using 3D to 2D design principles. The full sheet with its small-scale folded tissues is then folded using origami techniques to create an overall tissue (or organ) that mimics native structure.

In view of the wide variety of embodiments to which the principles of the present invention can be applied, it should be understood that the illustrated embodiments are exemplary only, and should not be taken as limiting the scope of the present invention.

Other aspects, modifications, and embodiments are within the scope of the following claims. The invention may be embodied in other specific forms without departing from the essential characteristics thereof. The present embodiments are therefore considered to be illustrative and not restrictive.

What is claimed is:

1. A method of forming 3D engineered tissues comprising:
a) providing a 2D scaffold material comprising a plurality of fold locations and a plurality of cell assembly sites;
b) assembling cells into the cell assembly sites; and
c) folding the 2D scaffold material along the fold locations to form a 3D scaffold structure.

3. The method of claim 2 wherein at least one of the predefined structures comprises a vascular pathway.

4. The method of claim 3 wherein the vascular pathway is formed between adjacent folded layers of the 3D scaffold structure.

5. The method of claim 3 wherein the vascular pathway is formed by a plurality of through holes in the 2D scaffold material that are aligned in the 3D scaffold structure to form the vascular pathway.

6. The method of claim 1 wherein the cells comprise at least two different types of cells.

7. The method of claim 6 wherein the cells comprise endothelial cells, fibroblasts and hepatic cells.

8. The method of claim 7 wherein the endothelial cells form vascular pathways in the 3D scaffold structure.

9. The method of claim 1 wherein the scaffold material comprises a biocompatible and biodegradable polymer.

10. The method of claim 9 wherein the scaffold material comprises a biocompatible and biodegradable polyurethane polymer or poly(polyol sebacate) polymer.

11. The method of claim 10 wherein the scaffold material comprises BDI-BDO-BDI-BDO-BDI/PCL thermoplastic polyurethane wherein BDI is 1,4-butanediisocyanate, BDO is 1,4-Butanediol, and PCL is polycaprolactone.

12. The method of claim 1 wherein the fold locations on the 2D scaffold material comprise thinned regions of the scaffold to facilitate bending.

13. The method of claim 1 wherein the 2D scaffold comprises integrated actuators that facilitate folding of the 2D scaffold material.

14. The method of claim 13 wherein the integrated actuator comprises a polymer bilayer in which one layer expands in liquid.

15. The method of claim 1 wherein the 2D scaffold comprises a fold pattern with a single degree of freedom thereby facilitating folding.

16. The method of claim 1 wherein assembling the cells comprises templated assembly by selective removal.

17. The method of claim 1 further comprising allowing the cells to coalesce and the scaffold material to biodegrade to produce a tissue unit.

18. The method of claim 17 further comprising forming a larger scale tissue by combining a plurality of tissue units in a modular fashion.

19. A 3D scaffold structure produced by the method of claim 1.

20. A tissue unit produced by the method of claim 17.
